(19) United States
(12) Patent Application Publication
(10) Pub. No.: US 2026/0069655 A1
(43) Pub. Date: Mar. 12, 2026

(54) METHOD OF TREATING NEUROINFLAMMATORY SYMPTOMS OF CENTRAL NERVOUS SYSTEM (CNS) DISORDERS BY ADMINISTRATION OF CHOLESTEROL DEPENDENT CYTOLYSINS (CDC)

(71) Applicant: RESOLYS BIO, INC., Delanson, NY (US)

(72) Inventors: Mitchell Sanders, Southborough, MA (US); John McMichael, Delanson, NY (US)

(21) Appl. No.: 19/107,503

(22) PCT Filed: Aug. 31, 2023
(86) PCT No.: PCT/US2023/031727

§ 371 Date: Feb. 27, 2025

Related U.S. Application Data
(60) Provisional application No. 63/403,227, filed on Sep. 1, 2022.

(51) Int. Cl. A61K 38/16; A61P 25/00; A61P 25/28

(52) U.S. Cl. CPC A61K 38/164; A61P 25/00; A61P 25/28

(57) ABSTRACT

Disclosed herein is a method of treating chronic and acute neuroinflammatory symptoms of a CNS disorder by administration of effective amounts of sublytic concentrations of a cholesterol dependent cytolysin (CDC).

### CROSS-REFERENCE TO RELATED APPLICATION

This application claims the benefit of priority to U.S. Provisional Patent Application No. 63/403,227, filed Sep. 1, 2022, the entire disclosure of which is incorporated herein by reference in its entirety.

### BACKGROUND

Trauma is the leading cause of death in individuals between the ages of 1-45 with traumatic brain injury (TBI) being the major cause (1.5 M cases with over 50,000 deaths annually in the United States alone and an economic impact of $80 billion (1)).

The damage from TBI is caused by direct or transmitted external physical forces: motor vehicle collisions, sport injuries, falls, assault, or pressure blasts (2). TBI can result in physical, cognitive, and behavioral impairments, leading to temporary or permanent dysfunction (3).

A traumatic brain injury (TBI) is a disruption of function in the brain that results from a blow or jolt to the head or penetrating head injury. TBI is heterogeneous in its cause and can be seen as a two-step event: 1) a primary injury, which can be focal or diffuse, caused by mechanical impact, that results in primary pathological events such as hemorrhage and ischemia, tearing of tissue and axonal injuries; 2) a secondary injury such as diffuse inflammation, cell death and gliosis, which is a consequence of the primary one. This secondary injury starts immediately after injury and can continue for weeks, and is thought to involve an active inhibition of neural stem cell activity. Collectively, these events lead to neurodegeneration.

A large fraction of TBI is mild, and thus may go undiagnosed immediately after injury. Undiagnosed and untreated TBI presents a risk because some signs and symptoms may be delayed from days to months after injury, and may have significant impact on the patient's physical, emotional, behavioral, social, or family status if untreated, and may result in a functional impairment. Because secondary damage from the injury continues after the initial impact, early treatment (and thus rapid diagnosis), particularly point-of-care treatment, is desirable. An ideal therapy for TBI would reduce the injury infarct size as well as limiting the secondary inflammatory responses. In the U.S., about 1.5 million people per year suffer a traumatic brain injury (TBI), reflecting physical damage to the brain that compromises brain function either temporarily or permanently. Of the total number so injured, some 50,000 die while another 80,000 have some degree of disability. The leading causes of TBI are accidents (auto, bicycle, pedestrian), assault, and sport-related injury.

Head injuries are described as being open or closed. Open head injuries involve penetration of the scalp and skull by bullets, sharp objects, or skull fractures resulting in laceration of brain tissue.

Closed injuries occur when rapid brain acceleration or deceleration results from shaking, crash, falls or other sudden insult. This rapid acceleration or deceleration can damage the brain at the point of contact (coup) or opposite that point (countercoup). The temporal and frontal lobes are most susceptible to damage, which can involve axon and/or blood vessel tearing. Torn blood vessels can leak and lead to hematomas, contusions, or intracerebral and subarachnoid hemorrhages.

Concussion is described as an immediate, but transient, loss of consciousness accompanied by a short period of amnesia. However, the TBI victim may appear to be dazed, disoriented or confused. A concussion may be accompanied by convulsions, hypotension, fainting and facial pallor. These signs and symptoms are usually short-lived in cases of single, uncomplicated concussion.

Traumatic brain injury can be both acute, occurring recently, as well as of the chronic form resulting from the long term consequences of such acute brain injuries including the effects of inflammation and scarring on the brain tissue.

Football and soccer players appear to suffer a significantly higher frequency of concussion than athletes in other sports. Professional football players in the National Football League (NFL) have recently brought to the public's attention the long-term consequences of multiple concussions incurred during their playing days. Included as frequently reported signs are loss of cognition, decreased communicative skills, compromised emotional stability, poor coordination, memory loss and dementia.

An increasingly prevalent subset of TBI is blast-induced or blast TBI (bTBI). With the increasing use of explosives, including improvised explosive devices (IEDs) in the global war on terrorism, bTBI is also increasing. Such injuries are often referred to as the hallmark injury of the wars in Iraq and Afghanistan, and affect both military and civilian workers in battle zones. Blast injuries are the most common cause of TBI in US soldiers in combat and a major cause of disability among service members.

Blast injuries can result in the full spectrum of closed and penetrating TBIs (mild, moderate, and severe). Mild and moderate TBI's are more prevalent than severe injuries in the current military conflict due to the vast improvement in protective gear, leading to an increase in survivors of bTBI.

Blast injuries are defined by four potential mechanism dynamics: (1) Primary Blast Atmospheric over-pressure followed by under-pressure or vacuum; (2) Secondary Blast Objects placed in motion by the blast hitting the subject; (3) Tertiary Blast: Subject being placed in motion by the blast and (4) Quaternary Blast: Other injuries from the blast such as burns, crush injuries, amputations, toxic fumes.

bTBI are typically closed-head injuries and are more complex than other forms of TBI, with multiple mechanisms of injury including shockwave transmission through the skull and sensory organs of the head. In a patient sample seen in the Department of Veterans Affairs (VA) polytrauma system, the pattern of injuries was different among those with injuries due to blasts versus other mechanisms. Injuries to the face (including eye, ear, oral, and maxillofacial), penetrating brain injuries, symptoms of posttraumatic stress, and auditory impairments are more common in blast-injured patients than in those with war injuries of other etiologies. Sayer N A et al. (2008) Arch Phys Med. Rehabil. January; 89:163-70. Accordingly there remains an interest in therapies which might be effective in treating one or more symptoms of both acute and chronic traumatic brain injury.

Related to but not limited to traumatic brain injuries are hematomas in the brain. Hematomas are broadly defined as collections of blood outside of blood vessels and when they occur in the brain they are associated with the activation and migration of microglial cells to the location of the injury.

The clinical severity of TBI has long been stratified according to post-resuscitation Glasgow Coma Scale scores into mild (GCS 14-15), moderate (GCS 9-13), and severe (GCS 3-8) (4,5).

TBI can be classified into primary and secondary injuries. Primary injuries occur at the time of the injury as a direct result of traumatic physical impact. Secondary injuries occur over hours and days, and generally result from complex biochemical processes that initially manifest as cerebral edema and elevated intracranial pressure (6).

The delayed nature of secondary injuries provides a glimmer of hope for using neuroprotective agents to interrupt apoptosis in vulnerable tissue, which is a major focus of drug development research in TBI. However, despite promising results in preclinical testing, a wide variety of neuroprotective agents have failed to provide any clinical benefit (6,7).

Although there have been decades of progress in preclinical data suggesting promising therapies for TBI, none of the candidates were efficacious in clinical trials reflecting the heterogeneity of the patients and lack of better diagnostic tools to predict clear outcomes. The failed pharmacological therapies include but are not limited to corticosteroids, progesterone, erythropoietin (EPO), amantadine, tranexamic acid (TXA), and citicoline (8-13). Additionally, most of the therapies targeted the acute phases of primary and secondary TBI injuries, while few looked at any new drug indications to reduce the long term/chronic manifestations of TBI including memory loss, improper balance, reduced language skills, headaches, seizures, dizziness, mood swings, or depression.

Streptolysin O (SLO) is one of a group of filterable hemolysins derived from Group A beta-hemolytic streptococci. Specifically, streptolysin O is a 60-kD peptide, which is hemolytic in its reduced state, but is inactivated upon oxidation (Johnson et al., Infect. Immun., 27:97-101, 1980; Alouf et al., Pharmacol. Ther., 3:661-717, 1984; Bhakdi et al., Infect. Immun., 47:52-60, 1985, the disclosures of which are incorporated herein by reference in their entirety) and can be designated as oSLO. Group A streptococci produce streptolysin O which in its reduced state has toxic effects. Once oxidized the lytic effects of SLO are substantially reduced or eliminated and such oxidation is effectively irreversible. Streptolysin O is used in the art generally as an analytical reagent for permeabilizing cells (e.g. Razin et al., Proc. Nat'l. Acad. Sci. (USA) 91:7722-7726 (1994).

Of interest to the present invention are the disclosures of U.S. Pat. Nos. 5,576,289 and 5,736,508 which are hereby incorporated by reference. U.S. Pat. No. 5,576,289 discloses the use of streptolysin O in methods for treating disease states characterized by motor deficit including multiple sclerosis and autism. U.S. Pat. No. 5,736,508 discloses the use of streptolysin O in methods for treating scarring.

Of further interest to the present invention are the disclosures in U.S. Pat. Nos. 7,196,058 and 7,629,058 the disclosures of which are incorporated by reference herein which disclose streptolysin O as having effects on MMP-2 which is a keratinocyte cell surface marker and is believed to be involved in the breakdown of extracellular matrix in normal physiological processes. Keratinocyte migration is involved in wound healing.

Of still further interest to the present invention is the disclosure of McMichael, U.S. Pat. No. 10,213,480 which discloses a method of inhibiting microglial cell migration in the brain of mammalian subjects suffering from TBI comprising the administration of Streptolysin such that microglial migration is inhibited. However, the mechanism of action is not well understood.

In parallel to this work on TBI, a group in Sweden and the UK were studying the pneumolysin (PNL) in an effort to develop novel peptide-based drugs that could inhibit Streptococcus infections 16-17, WO2022002982A1). They discovered that cholesterol dependent cytolysin, PNL binds the mannose receptor C type 1 on human dendritic cells (DCs) and murine alveolar macrophages. The PNL interaction mediates pneumococcal internalization to polarize native T cells into IFN-g^{low} IL4^{high} and FoxP3± immunoregulatory phenotype which makes the cells unable to illicit an innate inflammatory response. The researchers showed that mice pretreated with PNL were unable to fight off a pneumococcal infection due to the down regulation of the inflammatory response.

More recently a group in University of Maryland School of Medicine uncovered that TBI at both early and late stages alters dendritic cell (DCs) differentiation concomitant with a reduction of reactive oxygen species (ROS) levels in progenitor cells which may reflect why patients with acute and chronic CNS injuries such as TBI are prone to systemic infections and poorer clinical outcomes (19).

Tuszynski et al., in 2020 discovered that after injury, mature neurons in adult brains revert back to an embryonic state (20). This provides tremendous potential for finding drug candidates that can reactivate these embryonic like neurons to reverse the profound long-term side effects of TBI.

There remains a need in the art for improved methods for treating the chronic and acute neuroinflammatory symptoms of central nervous system disorders.

### SUMMARY

The present invention relates to the observation that cholesterol dependent cytolysins (CDCs) appear to reverse the acute and chronic side effects of central nervous system (CNS) disorders including traumatic brain injury (TBI). Without intending to be bound by any particular theory it is believed that the therapeutic effects of CDCs might be through a down regulation of the inflammatory response of antigen presenting cells (APC) and an alleviation of the deleterious effects of TBI. Also contemplated are modified CDCs produced by point mutations and chemical modifications of CDCs to reduce the hemolysis activity while retaining the therapeutic advantage of this class of new drug entities. Preliminary clinical evidence suggests that patients treated with CDCs have improved memory and reasoning skills suggesting that CDCs may play a role improving the synaptic strength and neuronal connectivity which are critical for memory formation.

Provided are methods for treating the chronic and acute neuroinflammatory symptoms of a central nervous system (CNS) disorder comprising administering an effective amount of a sublytic concentration of a cholesterol dependent cytolysin (CDC). Particularly preferred are streptolysin O (SLO) (including oxidized SLO (OSLO) and pneumolysin (PNL). According to one aspect of the method, the treatment restores CNS functions including attention and working memory, mental flexibility and speed, verbal reasoning, visual reasoning, verbal memory, motor and sensory balance and cognitive memory function. According to another aspect of the method the treatment reduces symptoms selected from the group consisting of headaches, seizures, dizziness, mood swings, and depression. According to an alternative aspect of the invention the CNS is not traumatic brain injury and according to a further aspect of the invention the CNS disorder is not associated with microglial cell migration.

Mutations with alanine or glycine in the transmembrane helices in Domain 3 (TMH1) (residues 259-288) and TMH2 (residues 359-386) result in inability of SLO to insert into the cell membrane and form a pore. These single or multiple point mutations in the amino acid sequence of CDC reduce the toxicity of these molecules by preventing hemolysis without reducing the beneficial activity of reprogramming APCs such as dendritic or neural progenitor cells to treat patients with chronic and acute TBI. Mutations in Domain 4 including but not limited to Cys 530 in the tryptophan rich undecapeptide loop result in the inability of SLO to properly interact with cholesterol preventing he proper stacking of streptolysin O monomers to form a pore.

Although (SLO) is easily oxidized (PNL) is much more potent. Data demonstrate the sublytic doses of a crude extract from Streptococcus pneumonia incubated with dendritic cells. The PNL extract requires at least a 1:1000 dilution of the extract to prevent hemolysis of dendritic cells.

PNL was purified by gel filtration using a Superdex 75/300 column and the purity seems to be >95% by SDS PAGE. The purified PNL was utilized in cell-based assays described below to examine the activation of dendritic cells.

In another embodiment, the cysteine residues of the undecapeptide region in Domain 4 of CDC are chemically modified through the reduction with dithiothreitol (DTT) and an alkylation agent including but not limited to iodoacetamide to reduce the hemolytic activity of CDCs while retaining the activity to treat patients with acute and chronic TBI.

Administration of the CDCs is at sublytic concentrations so as to not promote a cell lysis reaction in the subject being treated. Those of ordinary skill would be capable of determining such concentrations for particular CDCs and the subject being treated. According to a preferred aspect of the invention the use of CDCs which have been modified to reduce their lytic properties is contemplated. Thus, CDCs having one or more mutations in one of the short hydrophobic loops or the undecapeptide at the tip of domain 4 to prevent cholesterol binding and preventing hemolysis are contemplated as being particularly useful. According to another aspect of the invention the CDC has one or more mutations in domain 3 or domain 4 which inhibits the oligomerization of the membrane bound monomers or insertion of the beta sheets into the membrane thereby preventing hemolysis.

According to another aspect of the invention administration of CDCs restores memory and reasoning processes by improving the synaptic strength and neuronal connectivity which are critical to formation and recollection of memories.

### DETAILED DESCRIPTION

Methods are provided of using sublytic concentrations of cholesterol dependent cytolysins (CDC) (0.01-200 ng per dose) for modulating the homeostasis imbalance in acute and chronic phases of TBI by quelling the inflammation of antigen presenting cells (APC) and reprograming the neural plasticity of the neural progenitor cells (NPC) to reduce the deleterious symptoms associated with chronic disease (CDC) can reprogram antigen presenting cells (APC) resulting in a rebalance of dendritic cells (DC) to quell the acute and chronic inflammatory phases of tissue repair and remodeling after a traumatic brain injury (TBI).

### Example 1

According to this example human monocytes were incubated with IL-15 for 6-9 days to form mature dendritic cells, The cells were then treated overnight with serial dilutions of purified OSLO. After overnight incubation cells were harvested, and RNA was isolated. The concentration of RNA was determined and normalized to 20 μg/mL. Then, gene micro array analysis was done by using the NanoString nCounter Human Neuroinflammatory panel containing 770 genes and 23 pathways. Clinical doses of OSLO were tested to see their effect on the regulation of gene expression in mature human dendritic cells. The findings presented in Tables 1 and 2 below suggest that, in 48 hours of exposure to oSLO, statistically significant changes in gene expression occur. The biochemical pathways primarily involved include cytokine signaling, integrated stress response, autophagy and growth factor signaling. In particular, the results show that PLA2G4A, a gene associated with delirium, dementia, amnesia and cognitive disorders, and CD209 which is associated with psychogenic disorders are downregulated.

TABLE 1
Genes Down Regulated in Mature Dendritic
Cells treated with oSLO

| | | Fold | | |
|---|---|---|---|---|
| | Gene | Expression | p val | adj p val |
| Cytokines | CDKN1A | −1.75277 | 0.00248 | 0.0412 |
| | CSF2RB | −1.61613 | 0.0021 | 0.03768 |
| | IL3RA | −2.34666 | 0.00043 | 0.01915 |
| | LTA | −3.85214 | 1.80E−09 | 1.08E−06 |
| | TNFRSF1B | −1.54932 | 9.04E−06 | 0.0018 |
| Cognitive | PLA2G4A | −1.56876 | 0.00054 | 0.0201 |
| Disorders | | | | |
| Psychogenic | CD209 | −3.53637 | 0.00095 | 0.03008 |
| Disorders | | | | |

TABLE 2
Genes Up Regulated in Mature Dendritic Cells treated with oSLO

| | | Fold | | |
|---|---|---|---|---|
| | Gene | Expression | p val | adj p val |
| Integrated | ATG14 | 1.54819 | 0.00184 | 0.03554 |
| stress Response | CHEK2 | 1.60203 | 3.82E−07 | 0.00011 |
| | MFGE8 | 1.50773 | 0.00174 | 0.0347 |
| | TCIRG1 | 1.61031 | 0.00335 | 0.04375 |
| Growth Factors | TLR2 | 1.5616 | 0.00147 | 0.03381 |
| | PLD1 | 1.63333 | 0.00061 | 0.02157 |
| Others | DNMT3A | 1.61889 | 0.00065 | 0.02167 |
| | EZH1 | 1.50338 | 0.0035 | 0.04375 |
| | KLRB1 | 1.64899 | 0.00162 | 0.034 |
| | AK1 | 2.40741 | 2.17E−05 | 0.00325 |
| | NOD1 | 1.65914 | 0.00046 | 0.01915 |

### Example 2

According to this example, a 5 ml overnight culture of Streptococcus pneumonia was spun down and sterile filtered to remove the bacteria and then this crude PNL extract was serially diluted with PBS to determine the sublytic dose that can activate dendritic cells without hemolysis. Briefly, human monocytes were incubated with IL-15 for 6-9 days to form mature dendritic cells. The cells were then treated with serial dilutions of purified oxidized streptolysin O (OSLO) or PNL extract, and cell lysis was examined by phase contrast microscopy. No concentration of OSLO caused hemolysis. Whereas the PNL caused complete hemolysis at undiluted and 1:10, noticeable hemolysis at 1:100, and no hemolysis at 1:1000 and 1:10,000.

### Example 3

According to this example, it was demonstrated that PNL can have a safer dose range if reduced and alkylated. Specifically, a crude extract on PNL was treated with DTT for 10 minutes at 95° C. and then alkylated with IAA for 30 minutes in the dark and followed by incubation a second treatment with DTT prior to testing for cytolysin activity. Following this treatment cytolysin activity was dramatically reduced providing a safer dose range of PNL to activate antigen presenting cells.

### Example 4

According to this example, 14 patients with mild symptoms of TBI sought treatment under compassionate use. The patients who volunteered for the study had mild TBI with more than 12 different symptoms. The patients had an average age of 50 years and 75% of the participants were women. The patients were treated four times daily (QED) with a sublingual dose of the CDC streptolysin O for a period of eight to nine months. Post-treatment evaluations using a series of cognitive tests, referred to as the Meyers Neurological Battery (“MNB”, Meyers and Rohling, (2004) were compared to pre-treatment baseline evaluations. The comparative data are summarized in Table 3. Sublingual doses of OSLO were determined to have statistically significant (p val >0.05) improvements in reducing the chronic side effects of TBI related to visual/verbal memory and reasoning with a significant average of improvement of 13% OSLO was less effective at improving motor and sensory skills with a modest percent improvement of 5%.

These findings suggest that the neurons that are involved in storing memories, called engrams, may be reactivated after TBI through the treatment with sublytic doses of CDCs. Without being bound by any particular theory of invention it is believed that the CDC may improve the synaptic strength and neural connectivity which are thought to be a critical process for memory formation and recollection (Josselyn arid Tonegawa, 2020).

TABLE 3
Improved Memory and Reasoning Function in Patients
Treated with CDC Streptolysin O based on Meyers Neurological
Battery (“MNB”) Testing.

| Cognitive Memory Function | % Improvement |
|---|---|
| Attention and Working Memory: | 7% |
| Mental Flexibility and Speed: | 11% |
| Verbal Reasoning: | 13% |
| Visual Reasoning: | 12% |
| Verbal Memory: | 14% |
| Visual Memory: | 18% |
| Motor and Sensory: | 5% |
| ALL TESTS: | 11% |
| VVMR TESTS: | 13% |

Table 1. Average scoring of the cognitive functions of 14 patients presenting with mild symptoms of traumatic brain injury (TBI) after sublingual treatment with CDC for up to 9 months. The table provides a summary of the improvement of these patients in 7 major areas scored for during the Meyers Neurological Battery (“MNB”). The most noticeable improvements were in four areas representing memory and reasoning.

### Example 5

According to this example, a female suffering from traumatic brain injury was treated with pneumolysin containing drops for a month at the rate of one drop 3-4 times daily. Each drop contains 1.5 ng pneumolysin. She reported improved cognition, increased ability to find the right word during conversation, increased energy, and improvement in multi-tasking.

### Dosage

The dose of CDC administered to the subject can be determined by the physician, taking into account, age, sex, weight, etc. of the subject. In some embodiments, the streptolysin O is administered in a dosage amount ranging from about 0.00016 ng to about 3200 ng per dose. In some embodiments, the CDC is provide at a dose of about 0.016 ng, or about 0.02 ng, or about 0.05 ng, or about 0.1 ng, or about 0.2 ng, or about 0.3 ng, or about 0.4 ng, or about 0.5 ng, or about 1 ng, or about 10 ng, or about 20 ng, or about 40 ng, or about 60 ng, or about 80 ng, or about 100 ng, or about 500 ng, or about 1000 ng, or about 1500 ng, or about 2000 ng, or about 2500 ng, or about 3000 ng per dose. In some embodiments, the CDC is provided at a dose ranging from 0.016 ng to about 32 ng per dose or from about 0.16 ng to about 3.2 ng per dose.

In some embodiments, the administered dose of CDC is at least 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 65, 70, 75, 80, 85, 90, 95, 100, 110, 120, 130 or more International units. In one embodiment, the administered dose of CDC is about 1.6 ng (about 4 International units). In some embodiments, CDC is administered 1, 2, 3, 4, 5, 6, 7, 8, 9, 10 or more times daily for a period of 1, 2, 3, 4, 5, 6 or more weeks. Additional therapy may be administered on a period basis, for example, daily, weekly or monthly.

The term “effective amount” as used herein, refers to an amount of the therapy CDC sufficient to treat, ameliorate, or prevent the identified disease or condition (or symptoms associated with the disease or condition), or to exhibit a detectable therapeutic, prophylactic, or inhibitory effect. The effect can be detected by, for example, an improvement in clinical condition or a reduction in symptoms. The precise effective amount for a subject will depend upon the subject's body weight, size, and health; the nature and extent of the condition. Therapeutically effective amounts for a given situation can be determined by routine experimentation that is within the skill and judgment of the clinician.

The subjects treated in the methods disclosed herein in its many embodiments are desirably human subjects, although it is to be understood that the principles of the presently disclosed subject matter indicate that the presently disclosed subject matter is effective with respect to invertebrate and to all vertebrate species, including mammals, which are intended to be included in the term “subject.”

### Formulation and Route of Administration

The CDC described herein may be formulated in pharmaceutical compositions with a pharmaceutically acceptable excipient, carrier, or diluent.

In some embodiments, the pharmaceutical compositions are formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8. More particularly, the pharmaceutical compositions comprises in various aspects a therapeutically effective amount of at least one composition as described herein, together with one or more pharmaceutically acceptable excipients.

The term “pharmaceutically acceptable excipient” refers to an excipient for administration of a pharmaceutical agent, such as the compounds described herein. The term refers to any pharmaceutical excipient that may be administered without undue toxicity.

Pharmaceutically acceptable excipients are determined in part by the particular composition being administered, as well as by the particular method used to administer the composition. Accordingly, there exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, pH buffering substances, and the like. Liposomes are also included within the definition of pharmaceutically acceptable excipients.

Additionally, the pharmaceutical compositions may be in the form of a sterile injectable preparation, such as a sterile injectable aqueous emulsion or oleaginous suspension. This emulsion or suspension may be formulated by a person of ordinary skill in the art using suitable dispersing or wetting agents and suspending agents. The sterile injectable preparation may also be a sterile injectable solution or suspension in a non-toxic parenterally acceptable diluent or solvent, such as a solution in 1,2-propane-diol.

Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or diglycerides. In addition, fatty acids (e.g., oleic acid) may likewise be used in the preparation of injectables.

### PATENT CITATIONS AND REFERENCES

1. Vella M A, Crandall M L, Patel M B. Acute Management of Traumatic brain Injury, Surg Clin North Am. 2017 October; 97(5):1015-1030. doi: 10.101.6/j.suc.2017.06.003. PMID; 28958355; PMCID: PMC5747306.
2. Najem D, Rennie K, Ribecco-Lutkiewicz M, Ly D, Ilaukenfrers J, Liu Q, Nzau M, Fraser D D, Bani-Yaghoub M. Traumatic brain injury: classification, models, and markers. Biochem Cell. Biol. 2018 August; 96(4):391-406, doi: 10.1139/1342016-0160. Epub 2018 Jan. 25. PMID: 29370536.
3. Reis, C., Wang, Y., Akyol, O., Ho, W, M., li, R. A., Stier, G., Martin, R, and Zhang, J A I. 2015. 907 What's new in traumatic brain injury: update on tracking, monitoring and treatment. Int. J. 908 Mol. Sci. 16(6):11903-11965.
4. Hartings: J A, Bullock M R, Okonkwo D O, Murray L S, Murray G D, Fabricius M, et al. Spreading depolarisations and outcome after traumatic brain injury: a prospective observational study. Lancet Neurol. 2011; 10(12):1058-1064.
5. Teasdale G, Jennett B. Assessment of coma and impaired consciousness: a practical scale. Lancet. 1974; 304(7872):81-84
6. Puntis, M. and Smith, M. 2017. Critical care management of adult traumatic brain injury. 894 Anaesthesia and Intensive Care Medicine. 18(5):233-238.
7. Kochanek, P. M., Branlett, H. M., Dixon, C. E., Shear, D. A., Dietrich, W. D., Schmid, K. E., 706 Mondello, S., Wang, K. K., Hayes, R. L., Povlishock, J. T., and Tortella, F. C. 2016. 707 Approach to modeling, therapy evaluation, drug selection, and biomarker assessments for a 708 multicenter pre-clinical drug screening consortium for acute therapies in severe traumatic 709 brain injury: Operation Brain Trauma Therapy. J. Neurotrauma. 33(6):513-22.
8. Roberts I, Yates D, Sandercock P, Farrell B, Wasserberg J, Lomas G., et al. Effect of intravenous corticosteroids on death within 14 days in 10,008 adults with clinically significant head injury (MRC CRASH trial): randomised placebo-controlled trial. Lancet (London, England) 2004; 364(9442):1321-1328.
9. Wright D W, Kellermann A L, Hertzberg V S, Clark P L., Frankel M, Goldstein F C, et al. Protect: a randomized clinical trial of progesterone for acute traumatic brain injury. Ann Emerg Med, 2007; 49(4):391-402.
10. Skolnick B E, Maas A I, Narayan R K, van der Hoop R G, MacAllister T, Ward J D, et al. A clinical trial of progesterone for severe traumatic brain injury. N Engl J Med. 2014; 371(26):2467-2476.
11. Lee J, Cho Y, Choi K S, Kim W, Jang B H, Shin I I, et al. Efficacy and safety of erythropoietin in patients with traumatic brain injury: a systematic review and meta-analysis, Am J Emerg Med 2018; 37:1.101.
12. Hammond F M, Sherer M, Malec I F, Zafonte R D, Diktnon 5, Bogner J, et al. Amantadine did not positively impact cognition in chronic traumatic brain injury: a multi-site, randomized, controlled trial. 1 Weurotrauma. 2018; 35(19):2298-2305.
13. CRASH-2 Collaborators (Intracranial Bleeding Study) Effect of tranexamic acid in traumatic brain injury: a nested randomised, placebo controlled trial (CRASH-2 Intracranial Bleeding.Study)/11,17, 2011; 343: d3795
14. Zafonte R D, Bagiella E, Ansel B M, Novack T A, Friedewalci W T, Hesdorffer D C, et al, Effect of citicoline on functional and cognitive status among patients with traumatic brain injury: citicoline Brain Injury Treatment Trial (COBRIT) JAMA. 2012; 308(19):1993-2000.
15. McMichael J. US2018002860A1 Method of inhibiting microglial cell migration and treating traumatic brain injury. yAssignee Beech Tree Labs Inc.
16. Subramaniani K, and 0, Sotiriou. WO 2022/002982 AI, Mannose receptor-derived peptides for neutralizing pore-forming toxins for therapeutic uses.
17. Subratnanian K, Neill D R, Malak H A, Spelmink Imo, Khandaker 5, Dalla Libera Marchiori U, Dearing E, Kirby A, Yang M, Achour A, Wilvebrant 5, Nygren P A, Plant L, Kadioglu A, Henriques-Normark Pncurnolysin binds to the rnannosc receptor C type 1 (MRC-1) leading to anti-inflammatory responses and enhanced pneumococcal survival, Nat Microbiol. 2019 January; 4(I):62-70, doi: 10.1038/s41564-018-0280-x. Epub 2018 Nov. 12. PMID: 30420782; PMCID: PMC6298590.
18. Tsymbalyuk O, cierzanich V., Simard I M, Rathina.m C V. Traumatic brain injury alters dendritic cell differentiation and distribution in lymphoid and non-lymphoid organs bioR xiv 2021.12,28,474349.
19. Gunnar H. D. Poplawaki, Riki Kawaguchi, Erna Van Niekerk, Paul Lu, Neil Mehta, Philip Canete, Richard Lie, Ioannis Dragatsis, Jessica M, Meves, Binhai Zheng, Giovanni Coppola, Mark H. Tuszynski Injured adult neurons regress to an embryonic transcriptional growth state.islature.X20; DOI: 10, J.W8/01586-020-2200-5.
20. Meyers, J. E. & Rohling, M, L. (2004). Validation of the Meyers Short Battery on mild TBI patients, Archives of Clinical Neuropsychology, 19, 637-651.
21. Jocelyn S A, Tonegawa S, Memory engrams: Recalling the past and imagining the future. Science. 2020 Jan. 3; 367(6473):eaaw4325. doi: I 0.1126/science.aaw4325. PMID: 31896692; PMCID: PMC7577560.

Numerous modifications and variations in the practice of the invention are expected to occur to those skilled in the art upon consideration of the presently preferred embodiments thereof. Consequently, the only limitations which should be placed upon the scope of the invention are those which appear in the appended claims.

What is claimed is:

1. A method for treating the chronic and acute neuroinflammatory symptoms of a central nervous system (CNS) disorder comprising administering an effective amount of a sublytic concentration of a cholesterol dependent cytolysin (CDC).

2. The method of claim 1 where the CDC is streptolysin O or pneumolysin.

3. The method of claim 1 where the CNS disorder is traumatic brain injury (TBI).

4. The method of claim 1 where the treatment restores CNS functions selected from the group consisting of attention and working memory, mental flexibility and speed, verbal reasoning, visual reasoning, verbal memory, motor and sensory balance and cognitive memory function.

5. The method of claim 1 where the treatment reduces symptoms selected from the group consisting of headaches, seizures, dizziness, mood swings, and depression.

6. The method of claim 1 wherein the CDC is pneumolysin.

7. The method of claim 1 wherein the CNS disorder is not traumatic brain injury.

8. The method of claim 1 wherein the CNS disorder is not associated with microglial cell migration.
